(19) United States
(12) Patent Application Publication
(10) Pub. No.: US 2010/0016796 A1
(43) Pub. Date: Jan. 21, 2010

(54) Syringe Mechanism for Detecting Syringe Status

(75) Inventor: Kevin J. Derichs, Buda, TX (US)

Correspondence Address: JONES, WALKER, WAECHTER, POITEVENT, CARRERE;& DENEGRE, L.L.P., 5TH FLOOR, FOUR UNITED PLAZA, 8555 UNITED PLAZA BOULEVARD, BATON ROUGE, LA, 70809, US

(73) Assignee: ANIMAL INNOVATIONS, INC., Houston, TX (US)

(21) Appl. No.: 12/559,669

(22) Filed: Sep. 15, 2009

Related U.S. Application Data
(63) Continuation of application No. PCT/US2009/038098, filed on Mar. 24, 2009.
(60) Provisional application No. 61/039,158, filed on Mar. 25, 2008.

(51) Int. Cl. A61M 5/20; H01L 43/06

(52) U.S. Cl. 604/135; 324/207.2

(57) ABSTRACT

The syringe includes a body portion having an interior barrel chamber. The chamber has a fill port and a discharge port. An injection needle will be fluidly connected to the discharge port in use. The syringe includes a handle, having one portion that is moveable. Slidable in the barrel chamber is a plunger shaft, having a plunger on its terminal end. The trigger is operationally connected to the plunger shaft, so that movement of the trigger (or activation of the trigger in an electronic trigger embodiment) results in a corresponding movement of the plunger shaft. Connecting to the syringe are the fluid line, and a communications cable. Communications cable will include a power lead, if needed, and communications lines for communication between the syringe and the various computers.

### PRIORITY CLAIM

This application is a continuation of Patent Cooperation Treaty application number PCT/US2009/038098, filed Mar. 24, 2009, which claims the priority benefit of U.S. Provisional patent application No. 61/039,158 filed on Mar. 25, 2008.

### TECHNICAL FIELD

The present invention relates generally to a system and method to inject substances in animals using a syringe, and more particularly, to detect the position of the syringe plunger shaft and determine syringe status based thereon.

### BACKGROUND INFORMATION

It is often desirable to treat large numbers of individual animals, referred to herein generally as subjects, with a substance, such as a medication or other material, with speed, efficiency, and accuracy. At times it is desirable or necessary to mass vaccinate animals in the field or on a feedlot, such as vaccination of cattle in response to a condition or in response to a disease indicator. Additionally, the livestock industry requires routine vaccinating, medicating and/or treating of cattle or livestock. There are many diseases and illnesses contracted by livestock that need to be treated with various drugs and medications. Failure to properly treat the animals can result in significant losses to the rancher or feedlot or other party responsible for the livestock. Typically, the livestock is segregated into groups according to general size and weight. Often, the weight variation in a group of subjects is plus or minus 25% of the average weight of the group. Typically, the same amount of medication is administered to each of the subjects within a particular group. As a result, certain of the livestock are under-medicated while certain of the others are over-medicated, each situation presenting additional problems.

Multiple medications may be needed for administration in harsh dirty environments and under confusing adverse conditions. In these situations, medications must be delivered in large volumes and exposed to ambient conditions, with speed, efficiency, accuracy, and accurate maintenance of records. Treating with the correct dosage is considered necessary.

One system to address these problems is disclosed in U.S. Pat. No. 7,056,307 (the “'307 patent”), which is incorporated herein by reference in its entirety. This system discloses a highly accurate pump, reservoir bottle and syringe, where the pump delivers precise dosages of fluid medication from the reservoir bottle to the syringe for delivery. A central system computer determines the dosage to be injected to a given animal (generally determined by animal weight, medical history, medication records, and other pertinent information), and instructs the pump controller (which can be a computer or controlled by a computer) to dispense the calculated amount of fluid to a syringe. The pump then dispenses the proper amount of fluid (possibly in several doses, under the control of the system computer) to the syringe for injection into the animal by an operator or technician. The '307 system, or a comparable dosage controlled injection system, provides for ready administration of medications in accurate amounts and time-stamping the administration of the dose.

The system disclosed in the '307 patent provides an efficient system for dispensing medication to a syringe, under computer control, that provides an automatic dosing syringe system that is highly accurate and dependable. The system is capable of dispensing a variety of substances and be capable of operating in a wide range of ambient temperatures. The system is capable of having the syringe adapted to be automatically filled with the proper amount, under computer control. The syringe of the '307 system can be easily emptied, cleaned and disinfected without wastage of the medications. However, the '307 system does not provide for extensive feedback to the central computer system, particularly of the amount of stored material in the syringe that can be calculated based upon syringe plunger position, and more particularly, when the syringe is discharged, and ready to be filled with another dose. One system disclosed in the '307 patent to determine the status of the syringe is a hall effect sensor that interfaces with a fixed location magnet. However, the hall effect sensor is subject to becoming contaminated, particularly in the feedlot environment, and providing unreliable readings. Particularly, in the prior embodiment, the hall sensor is located in the plunger shaft, and (a) contamination of the shaft can present problems in detecting the magnet through a contaminated shaft; (b) detection of the magnetic field through the metal plunger shaft is problematic; (c) read filled syringes are not feasible with this design; (d) multiple magnets were not feasible. A more robust method is needed to detect the status of the syringe, particularly the status of the syringe as “discharged” and available for filling, or other possible status of the syringe.

### SUMMARY OF THE INVENTION

The objects, advantages and features of the invention will become more apparent by reference to the drawings which are appended hereto and wherein like numerals indicate like parts and wherein illustrated embodiments of the invention are shown, in which:

### BRIEF DESCRIPTION OF THE DRAWINGS

FIG. 1 is a diagrammatic sketch of a prior art weight dependent, automatic filling dosage system.

FIG. 2 is a cross section of a prior art syringe.

FIG. 3 is a perspective view of a syringe plunger with indicia

FIG. 4A is a side perspective view of one embodiment of the sensor housing.

FIG. 4B is a rear view of the sensor housing shown in FIG. 4A.

FIG. 5 is a diagrammatic block diagram showing some of the components that may be located in the sensor module

FIG. 6 is a perspective view of one embodiment of the syringe showing the sensor module and base module.

### DETAILED DESCRIPTION OF THE PREFERRED EMBODIMENT

It is to be understood that while the present invention is described below with respect to being used to administer an exact dosage of a substance to a subject such as an animal, the present invention is not limited to this type of application. The present invention may also be used in other applications, including administering injections to humans.

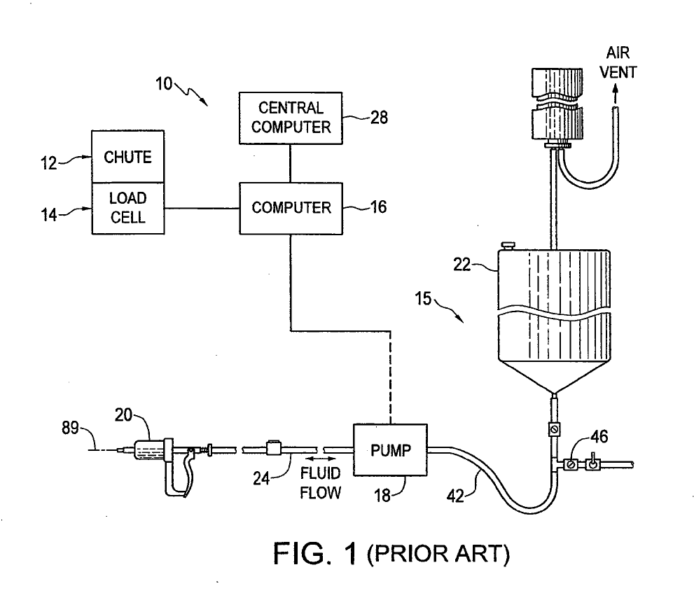

Referring to FIG. 1, a prior art dosage system in one embodiment of the present invention, generally referred to as 10, includes the arrangement and combination of several separate components. The dosage system 10 is used in conjunction with a restraining device 12 and a weighing device 14 as shown in FIG. 1. The restraining device 12 may be a squeeze chute used to secure the animal, and the weighing device 14 may be an electrically-operated load cell used to weigh the animal. The load cell 14 may include a digital output which is transmitted to and read by a central computer 28, or to a system microprocessor-based control device or computer 16 which communicates to a central computer 28. The central computer 28, or combination of computers 16 and central computer 28, are considered the system computer. It is to be understood that the output of the load cell 14 could also be an analog output.

With reference to FIG. 1, the dosage system 10 includes a plurality of units 15, with each unit 15 including a pump 18, syringe 20 and reservoir 22. Each unit pump 18 is in fluid communication with the unit reservoir 22 and the unit syringe 20. The syringe 20 is directly connected to the pump 18 via a first connecting tube 24. The pump 18 is connected to the reservoir 22 via a second connecting tube 42. Each sub-combination assembly of pump 18, syringe 20, reservoir 22, and connecting fluid lines 42 generally comprises the base injection unit 15. The number of units 15 in the automatic filling dosage system 10 may be determined by how many substances, drugs or chemicals are desired to be available for administering, i.e. one unit for each substance, drug or chemical. For example, a dosage system 10 may include just a single syringe unit, or many units.

In addition to the system computer receiving weight information on the animal from the load cell 14, the system computer may also have stored the animal's health-related information, to assist in activating the appropriate system units 15 for delivery of the proper medication to filling the appropriate syringe(s) 20 with the appropriate antibiotic/chemical at the correct drug dosage, from a fluid container receptacle 22. Valves 44 and 46 are used to control fluid movement. The system unit pump 18 shown is a valveless, substantially viscosity-independent pump, but other types may be used.

The system computer generally calculates the proper doses of the substances for the animal according to its weight. The system computer 16 signals the proper pumps 18 of each active station to fill the required syringes 20 according to the calculated dosage, thus filling the syringe. As the syringe handle is depressed, the fluid is administered and the syringe 20 is emptied. Upon the syringe 20 being emptied, a signal may be sent to the system computer that the syringe 20 is empty (for instance, a trigger switch may detect the handle as fully depressed). Upon release of the handle, the trigger switch status can be used by the system computer to determine that the syringe is available for refill. The above procedure will then be repeated as needed.

The treatment administered to the animal may be automatically recorded on the system computer to maintain current medical records on each of the animals. Alternatively, the medicine or chemical dosage for any animal that is calculated by the computer could be automatically recorded on a memory device of a chute side computer 16 whereby it could be later permanently documented.

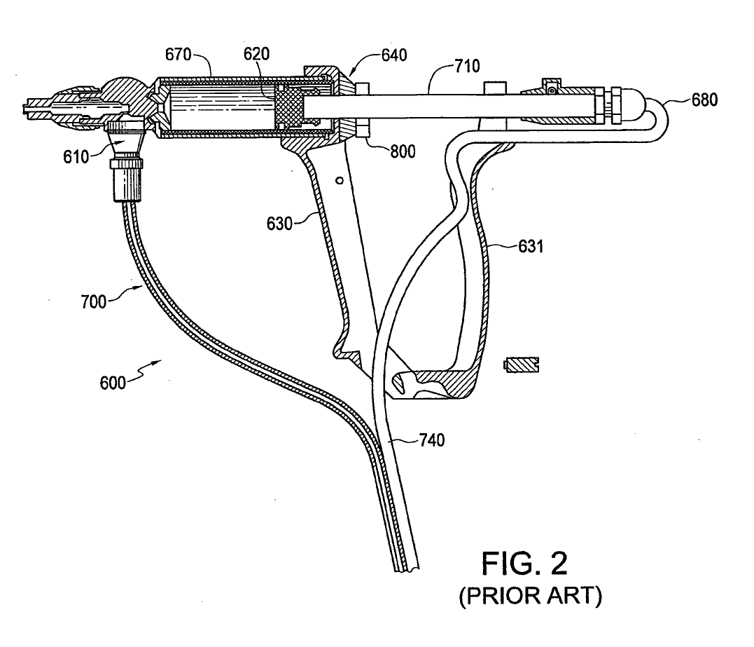

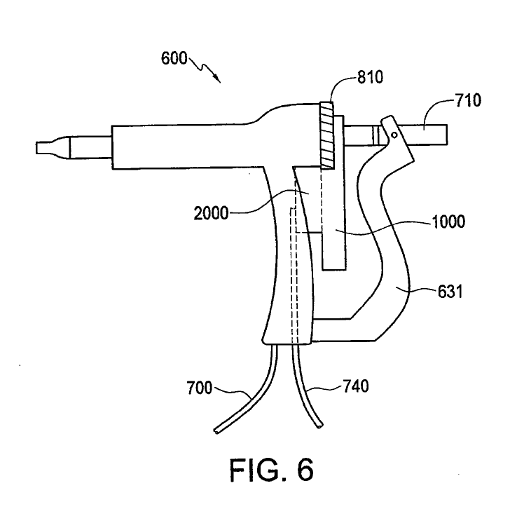

One prior art syringe 600 is shown in FIG. 2. The syringe 600 allows precise filling using a, for example only, spool valve assembly 610. The valve 610 allows medicament to enter the syringe from the pump 18 through fill tube 700. The syringe includes a body portion having an interior barrel chamber 670. The chamber 670 has a fill port/discharge port (the device may have a separate fill and discharge ports). An injection needle will be fluidly connected to the discharge port in use. The syringe includes a handle (630, 631), having one portion that is moveable (the trigger) 631. As shown in FIG. 2, the trigger 631 in this embodiment is the rearward most portion of the handle. Slidable in the barrel chamber is a plunger shaft 710, having a plunger 630 on its terminal end. The trigger 631 is operationally connected to the plunger shaft, so that movement of the trigger (or activation of the trigger in an electronic trigger embodiment) results in a corresponding movement of the plunger shaft 710. As shown, the trigger 631 is pivotally connected to the rear portion of the plunger shaft 710. Connecting to the syringe are the fluid line 700, and a communications cable 740. Communications cable will includes power lead, if needed, and communications lines for communication between the syringe and the various computers in the system. Communicating lines/power lines are routed as needed through the syringe, here shown terminating at the rear of the plunger guide 680. It is preferred that the incoming cables and hardware (such as plugs) not be positioned on the trigger portion of the handle to avoid cable/line failures. For instance, as shown in FIG. 6, the cables enter the syringe through the fixed portion of the handle.

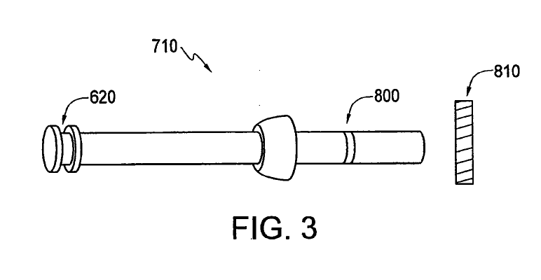

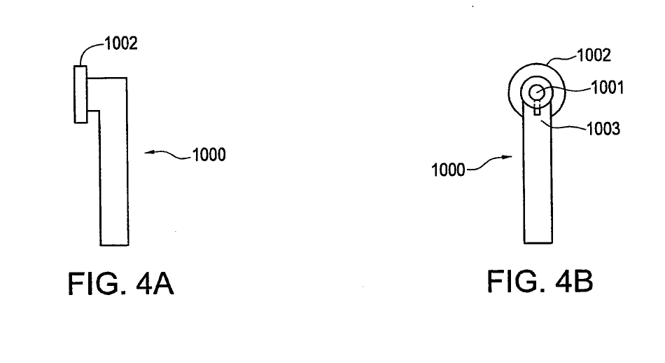

The medicine flows from the valve 610 into the barrel 670. By manually compressing the trigger 631, the trigger 631 drives the plunger shaft 710 inwardly, and consequently the plunger 620 forces medicament to be discharged from the barrel 670 through the valve 610 and out of the discharge port of the syringe 600. The single valve 610 takes the place of multiple valves in that it helps control the flow of medicament both into and out of the syringe 600, although multiple valves can be used. An embodiment of the plunger shaft 710 is shown in FIG. 3. Shown on the shaft is plunger end 620, plunger guide 640, and one position indicia 800. Plunger guide 640 has a center opening in which the plunger shaft 710 is slidable. The guide 640 functions to guide the plunger shaft in operation, and to seal the rear of the syringe about the plunger shaft. Plunger shaft 710 is retained to the syringe body by a retainer ring 810. The plunger is inserted into the barrel 670 of the syringe, and the locking ring 810 then slid over the shaft, and threads onto the syringe at the rear of the barrel 670. Other types of syringe systems can be utilized, such as the rear filling syringe system shown in FIG. 4 of the '307 patent.

Shown on the plunger shaft 710 is a position indicator 800. As shown, position indicator can be a magnetic region on the plunger shaft, which can be detected by a hall effect sensor positioned in the sensor module. Alternatively, the indicia may be an optically detectable region(s) (e.g. a reflective region or an opaque dark region) on the plunger shaft 710. This optically detectable region will be detected by an optical sensor located in a sensor module 1000, next described. The shaft 710 may have a series of indicia 800. In the case of an optically detectable region, each indicia may be more complex than a single bar—for instance, each indicia may be an encoded series of bars readable by a optical sensor such as an infrared bar code reader or scanner. As shown in FIG. 3, the indicia encircles the shaft 710, to accommodate possible rotation of the shaft in use. This arrangement is not needed if the shaft 710 is not rotatable (for instance, the shaft has a guide channel that interfaces a guide tab positioned in the barrel).

With reference to FIGS. 4A and 4B, a sensor module housing 1000 is depicted. The sensor module housing 1000 (sometimes referred to as a lollipop) is a plastic housing that contains the modules electronic components. The housing 1000 has a center opening 1001 into which the plunger shaft is slidably positioned. Surrounding the center opening is a mounting flange 1002. Sensor module is removably fixed to the plunger shaft 710 by retaining ring 810. The exterior housing of the sensor module may have to be notched to allow the retaining ring 810 to be slid over the bottom end of the housing casing into a locking position around the mounting flange 1002. To prevent rotation of the sensor module 1000 housing about the plunger shaft 710, the housing may be clipped, screwed or other wise attached to the syringe body. The sensor module housing has a transparent portion 1003 facing the center opening 1001 to allow the plunger shaft 710 to be viewed by the optical scanner in the sensor module. The sensor module center opening 1001 is preferably lined with a resilient material, such as a silicon o-ring, that acts to wipe and clean the plunger shaft 710 as it slides through the module opening. While the sensor module does not need to encircle the plunger shaft in order to “view” the plunger shaft, this is preferred as the working environment can quickly contaminate the plunger shaft, providing possible false readings. Encircling the shaft with the sensor module allows the shaft to be cleaned upon each stroke, reducing false readings.

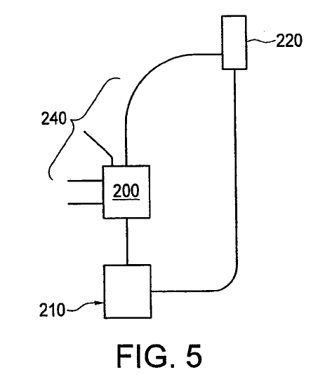

The electronic components in the sensor module 1000 are shown in a block format in FIG. 5. A processor or microprocessor 200 is located in the module housing (generally all component with be mounted on circuit boards in the interior of the housing). Operationally connected to the microprosessor is a sensor, hereafter described as an optical sensor (an emitter and receiver), preferably an infrared detector/emitter (a suitable optical sensor is the Optex OPB707A, available for Optek Inc. of Carrollton, Tex.). The optical sensor 220 illuminates and detects a return optical signal through an sensor opening 1003 in the module housing (preferably covered with a clear shield to protect the electronic components therein from contamination). Other discrete components may be included in the sensor module are a radio transmitter 270, and a modulator/demodulator 280.

As depicted in FIG. 4b, the sensor opening faces the plunger shaft 710 to view the indicia 800 located on the plunger shaft 710. The optical sensor 220 emits light and detects the light reflected off the plunger shaft 710 in order to detect the presence or absence of indicia 800. The operation of the sensor 220 is controlled by the microprocessor 200 (e.g. controls the power on/off sequence, and polling of the sensor output signal, to enable power conservation). The output signals from the sensor are received by the microprocessor 200 over communication lines 241, or may be directly routed to the central computer system (hard lined or via RF transmission). The status of the syringe can be determined from the output signal of the sensor 220. The output signal may simply reflect the presence or absence of an indicia (for the optical case) or to a signal whose strength is dependent upon the distance between the indicia and the sensor (for the hall effect case). The microprocessor may correlate the received output signal with a syringe status (charged, discharged, 10% full, etc) and communicated the “status” of the syringe to the system computer over communications lines 240, or via radio link via the base module, later described.

The microprocessor 200 may control or interface other components located in the sensor module or on the syringe, such as status indicators (e.g. an onboard displays, LEDs), switches (e.g. a trigger sensor), or other desired components. The display indicators can be used to provide a “syringe status” indicator to the user, such as syringe discharged, syringe charged, syringe malfunction, and other pertinent information. A LED display screen is not preferred due to the field environment conditions that the syringe may encounter in use.

The electronic components are powered via a power source 210, which may include an onboard battery, or direct electrical connections to a power source (such as through the communication/power cable to the syringe), or both, for instance, employing an onboard re-chargeable battery and external power source, with onboard battery charged by the external power source (for instance, using rectifiers if an external AC power source is used). Additionally, power may be provided to the sensor module indirectly through inductive coupling of resonant coils, one in the module, and another powered coil located external to the module, but sufficiently close for inductive coupling and power transfer, as later described.

Also contained in the sensor module is a communication means to communicate information to an external system, such as the central computer of the '307 system. The communication means may include hardwired communication lines 240 that couple with communications lines entering the syringe via cable 740, or an onboard radio system controlled by the microprocessor (or embedded in the microprocessor, such as the Microchip frPIC12F675F, from Microchip Technologies of Chandler, Az) that communicates with a remote radio receiver. Alternatively, the communication line may also be achieved by inductive coupling of resonant coils.

As the sensor module housing is removable with the plunger shaft, there are a number of ways to operationally couple the sensor module to the syringe. Several different embodiments will be described.

### Embodiment 1

### Direct Plug-in Coupling

To “connect” the module to the communication and power cables entering the syringe body, the sensor module may terminate with a plug, socket, wiring harness interconnect or other electrical connector, such as a terminal block, that interfaces with a corresponding electrical connector on the syringe, such as plug in location in the bottom of the sensor handle. This arrangement provides for direct communication/power to the sensor module, and allows the module to be removed from the syringe when the syringe needs to be cleaned. However, the plug contacts can become contaminated.

### Embodiment 2

### Base Module Electrical Contacts

Instead of a plug, the electrical/communication wires can terminate in a separate enclosed module, the base module 200 (see FIG. 6), and the base module 2000 interfaces with the sensor module via contacts on each module that interface, such as spring loaded pogo contacts and receivers (available from Everett Charles Technologies of Pomona, Ca, as the BIP-1 connector and receptacle) or other electrical contacts (plug/socket for instance). The base module 2000 could be removably fixed to the syringe body (snap in, screws, etc) or hard fixed to the syringe body (e.g. epoxied thereto), with the sensor module 1000 removably connectable to the base module 1000. For instance, the sensor module 1000 may be rotated about the plunger shaft until it aligns with the base module 2000 and the electrical contacts are made, or the sensor module may be slid along the plunger shaft 710 until in contact with the base module 2000. To removably fix the modules in a operating relationship, the two modules may be removably fixed to one another, such as through the action of a locking clip, screw attachment, or other means. The dual modules with corresponding contact points still presents the potential for contamination of the contact points, potentially disabling the machine

### Embodiment 3

### Coupled with Inductive Coils

An alternative arrangement is to use inductive coupling of the two modules for both power and communication signals. In this embodiment, the base module 2000 may contain an inductive coil and core, which inductively interfaces a corresponding resonating coil in the sensor module 1000. As the base and sensor modules are separated by substantially only the thickness of the housings of the two modules, coupled signals can be effective means to communicate, by modulation of the signal induced between the coils. Suitable modulators/demodulators circuits will be required in the appropriate module in this instance. With this arrangement, as physical contacts are lacking between the modules, and hence contamination is not an issue. The sensor module may be powered by a battery, and the battery may be charged by power transmitted by the induced signal (this will generally require rectifiers in the sensor module to condition the power for use).

### Embodiment 4

Alternatively, the sensor module may communicate with the base module wirelessly, such as through an RF transducer (or transceiver if two way communications are needed) and inductive coupling may be used only to provide power to the sensor module (e.g., the induced signal used to charge a capacitor or battery). This is preferred when coils are used, as modulating the induced signal increases the hardware needs and complexity of the device.

As described, all four embodiments have a means to receive power. In the first embodiment, external power is provided via the communications cable, while in embodiments 2 and 3, the power is provided via the base module, either direct electrical contacts, or inductive coupling. As indicated, the sensor module may be solely battery powered, using no external power, but his is not preferred.

If two way communications are needed (e.g. the ability of the system computer to send signals or commands to the sensor microprocessor), a single coil can be used employing different frequencies for the separate communications path, or alternatively, two pairs of inductive coil may be utilized for separate communications paths are desired.

With a two modules operationally coupled by induction, it is preferred that the base module include its own processor for routine tasks not required to monitor plunger shaft position, such as vibration indicator (a device to vibrate the handle to inform the user of syringe status (e.g. ready for use)), trigger indicators, or other functions. It is preferred that the functionally be contained as much as possible in the base module 1000, to reduce the required power transfer via inductive coupling in the sensor module 1000.

An example of use of the system using a series of indicia on the plunger shaft 710 for optical pickup will demonstrate the flexibility of the system. At any given time, the position of the plunger shaft is known or approximated (e.g. at 25% fill, or between 25-50% fill). Movement of the plunger shaft may be detected by the sensor system (by detecting the passage of indicia past the sensor) or from other status information that is known to either to the microprocessor (e.g. trigger depressed, or trigger not depressed) or the central computer (system filling, pump is activated). For instance, using magnets and a hall effect sensor, with proper orientation of the hall sensor, one could determine direction of movement based on whether the north or south pole is detected first. With the fixed sensor of the current invention, this is feasible as the magnet can completely traverse across the sensor, allowing detection of both the “N” and “S” poles. An example with optical indicia is where the status of the syringe may be confirmed as discharged by the sensor. In this instance, the plunger will be fully depressed, causing a properly positioned optical readable indicia on the shaft that corresponds to the plunger shaft fully inserted into the bore to be aligned with the optical sensor in the sensor module (or the magnet indicia, for instance, is aligned with the hall effect sensor). The sensor module detects the indicia, and directly or indirectly informs the central computer, through the communications link of the status of the syringe, here the status of discharged. Indirectly informing the computer could be informing the central computer of the actual output signal received from the sensor, which would allow the central computer to correlate such with a syringe status (e.g. 10% charged), while directly informing would be for the onboard module system to correlate the output signal of the sensor module (possibly using the previous stored reading to correlate a change in status) with a sensor status as “discharged” or otherwise, and to and communicate the correlated syringe status to the central computer over the communications lines. The “discharge” indicia may be the only indicia on the shaft needed; however, the optical system allows other indicia to be placed on the shaft to that correlates with the position if the plunger, and hence, the volume of the medication loaded in the syringe. For instance, in a syringe having a fully loaded chamber volume of 10 cc, the plunger may have five indicia to indicate the position of the plunger that corresponds to 2.5 cc charge increments, i.e., 0.0 cc (discharged), 2.5 cc (25% charged); 5.0 cc (50% charged); 7.5 cc and 10.0 cc charged. Increments can be set on the plunger as needed. Each indicia may unique, or simply be a single line (non-unique). With non-unique indicia, the relevant computer processor can correlate the status of the syringe by counting the number of indicia detected with the direction of movement of the plunger to arrive at a status value (e.g. 2.5 cc filled, between 2.5-5.0 cc's filled), starting from a known position that is stored in the system. The indicia values may be recorded in the module (e.g. microprocessor) or in the system computer, or elsewhere as needed. The direction of plunger shaft movement can be inferred from other status indicators located on the syringe, such as a trigger status switch (trigger depressed, syringe discharging), or from the system computer's information (syringe being loaded with medication). Instead of multiple optical indicia, multiple magnetic indicia can also be employed when using the Hall effect sensor.

In this fashion, the syringe can detect the location of the plunger shaft, and communicate that status to the system computer and use the status to set indicator lights (such as LED's) onboard the syringe. The ability to provide “syringe status” as detected at the syringe itself (the “onboard” status), allows this information to be communicated to the system computer for comparison with the status as determined by the system computer from other means (such as by counting the pump revolutions for load status, as provided for in the '307 application). If the onboard status and computed status are outside of a predetermined tolerance range, a warning may be given to the operator of a system problem (such as by lighting an indicator LED light on the syringe), indicating that the syringe requires service and should not be used. A status corresponding to system malfunction would be appropriate in this instance. For instance sometimes the motor turns and the motor position sensor responds back to the computer that is has turned, but due to air in the syringe fill line (which is compressible) the plunger may advance erratically, or not at all, again indicating a system or syringe malfunction.

The system as described provides a redundant method to verify the status of the syringe. The system is compact and robust, and is flexible for use in a highly contaminated environment.

1. A syringe injection device comprising a syringe having a body portion with an interior barrel chamber, said chamber having a fill port and a discharge port, said fill port fluidly connectable to a fluid reservoir, a plunger shaft slidably moveable in said chamber, the syringe including a handle having trigger, said trigger being operationally connected to said plunger shaft to move said plunger shaft in response to said trigger, said plunger shaft having a position indicia positioned thereon, a powered sensor module coupled to said syringe, said sensor module having a sensor for detecting said indicia, said sensor generating an output signal, said sensor module adapted to output a status of said syringe based upon the output of the sensor though a communication line.

2. The syringe of claim 1 wherein said indicia is a magnet, and said sensor comprises a hall effect sensor

3. The syringe of claim 1 wherein said indicia comprises an optically detectable indicia, and said sensor comprises an optical transmitter and an optical detector.

4. The syringe of claim 1 wherein said indicia comprises a plurality of indicia positioned on said plunger shaft.

5. The syringe of claim 1 wherein said sensor module further comprises a housing, said housing having an opening there through, and said plunger shaft being slidable in said housing opening.

6. The syringe of claim 1 wherein said sensor module includes a battery.

7. The syringe of claim 1 further having a powered base module, wherein said sensor module further has a means to receive power from said base module.

8. The syringe of claim 7 wherein said means to receive power comprises an inductive coil positioned in said based module and a corresponding inductive coil positioned in said sensor module.

9. A method of determining the status of a syringe, where said syringe has a body portion having an interior barrel chamber, a plunger shaft slidably moveable in said chamber, said plunger shaft having a position indicia positioned thereon, a powered sensor module coupled to said syringe, said sensor module having a sensor for detection of said position indicia, said sensor generating an output signal when activated, said sensor module further having a communication line for signaling to a remote computer a status of said syringe based upon the output signal of said sensor, said method comprising activating said sensor to detect said indicia or the absence of said indicia, and sensor outputting a signal, and communicating with said remote computer a status of said syringe based upon said output signal of said sensor.

10. The method of claim 9 wherein said status communicated comprises the output signal of said sensor.

11. The method of claim 9 wherein said sensor module further has a processor receiving said output signal from said sensor to determine a status of said syringe, said processor communicating with said remote computer, wherein the said processor communicates said status of said syringe to said remote computer.

12. The method of claim 11 wherein said status communicated comprises the status of “syringe discharged.”

13. The method of claim 12 wherein said status communicated comprises the status of syringe “charged.”

14. The method of claim 12 wherein said status communicated comprises the status of malfunction.
